SEQUENCE LISTING (5 sequences)

SEQ ID NO: 1 - protein, 263 aa, synthetic construct
  REGION 1..263: note = anti-CD19 scFV
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 45 aa, Homo sapiens
TTTPAPRPPTPAPTIASQPLSLRPEACRPAAGGAVHTRGLDFACD

SEQ ID NO: 3 - protein, 24 aa, Homo sapiens
IYIWAPLAGTCGVLLLSLVITLYC

SEQ ID NO: 4 - protein, 62 aa, Homo sapiens
KKVAKKPTNKAPHPKQEPQEINFPDDLPGSNTAAPVQETLHGCQPVTQEDGKESRISVQERQ

SEQ ID NO: 5 - DNA, 1185 bp, synthetic construct
  misc_feature 1..1185: note = Nucleic acid coding for the intracellular domain of a CAR of the invention
  source 1..1185: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtga

(19) United States
(12) Patent Application Publication
(10) Pub. No.: US 2025/0222106 A1
(43) Pub. Date: Jul. 10, 2025

(54) MYELOID CELLS MODIFIED BY CHIMERIC ANTIGEN RECEPTOR WITH CD40 AND USES THEREOF FOR ANTI-CANCER THERAPY

(71) Applicants: INSTITUT CURIE, PARIS (FR); INSTITUT NATIONAL DE LA SANTE ET DE LA RECHERCHE MEDICALE, PARIS (FR)

(72) Inventors: Philippe BENAROCH, PARIS (FR); Jovan NIKOLIC, MONTROUGE (FR)

(21) Appl. No.: 18/853,661

(22) PCT Filed: Apr. 7, 2023
(86) PCT No.: PCT/EP2023/059318

§ 371 Date: Oct. 3, 2024

(30) Foreign Application Priority Data
Apr. 7, 2022 (EP) 22305474.3

(51) Int. Cl. A61K 40/42; A61K 40/17; A61K 40/31; A61P 35/00; C07K 14/725; C07K 16/28

(52) U.S. Cl. CPC A61K 40/4211; A61K 40/17; A61K 40/31; A61P 35/00; C07K 14/7051; C07K 16/2803; C07K 2317/622

(57) ABSTRACT

A modified myeloid cell comprises a chimeric antigen receptor (CAR), or a modified induced pluripotent stem cell (iPS) or hematopoietic stem cell (HSC) comprising a CAR, wherein said CAR comprises an extracellular antigen-binding domain which binds to a tumor antigen or a tumor microenvironment (TME) antigen; a transmembrane domain; and an intracellular signaling domain comprising the CD40 cytotail. Therapeutic uses of the modified myeloid cell are disclosed.

The present invention concerns a modified myeloid cell comprising a chimeric antigen receptor (CAR), or a modified induced pluripotent stem cell (iPS) or hematopoietic stem cell (HSC) comprising a CAR, wherein said CAR comprises an extracellular antigen-binding domain which binds to a tumor antigen or an antigen present on cells of the tumor microenvironment (TME); optionally a hinge region; a transmembrane domain; and an intracellular signaling domain comprising the CD40 cytoplasmic tail. It also relates to therapeutic uses thereof.

Solid tumors and their metastases are the most common and therapeutically challenging types of cancer today. The tumor microenvironment (TME) is a complex, heterogeneous mix of cellular populations that interact with one another and with the tumor cells. The TME is immunosuppressive, both evading the immune system and preventing therapeutic intervention from efficiently eliminating malignant cells. Myeloid cells within the TME play an important role in contributing to immune evasion by exhibiting potent immunosuppressive as well as pro-tumorigenic properties.

TAMs (tumor-associated macrophages) are a key cell component of the TME in a variety of cancers. The prevailing consensus is that tumor-derived cytokines direct myeloid cell recruitment at the monocyte stage, and then the TME influences their development into polarized macrophages. TAMs can represent a significant portion of the tumor mass, up to 50% in some breast tumors. They develop into immunosuppressive macrophages, which hinder anti-tumor CD8⁺ T cells from infiltrating the tumor and attract or induce regulatory T cells (Treg). TAMs secrete growth factors like VEGF or TGFβ, which promote tumor growth and invasive behavior. They are generally associated with poor prognosis, though recent studies have shown that their impact on prognosis can vary depending on their localization and polarization (Ramos et al, 2022, Cell 185, 1-19, Tissue-resident FOLR2⁺ macrophages associate with tumor-infiltrating CD8⁺ T cells and with increased survival of breast cancer patients).

Impressive successes have been recently obtained to treat certain malignancies with autologous immune cell-based therapies. The most advanced approaches rely on T lymphocytes that have been genetically modified to express chimeric receptors that combine antigen-binding and T-cell activation activities in a single receptor are known as CAR T cells. Adoptively transplanted CAR T cells have shown considerable promise in fighting hematological malignancies.

CAR T cell treatments, however, have so far failed to treat solid tumors. These failures probably result from a combination of factors. First, identifying antigens strictly tumor-specific remains difficult, raising concerns about potential off-target effects. Second, before reaching the cancer cells within the tumor tissue, the CAR T cells may encounter physical barriers in the form of TAMs and cancer-associated fibroblasts that produce vast amounts of extracellular matrix. Third, CAR T cells do not successfully invade the TME due to a lack of metabolic resources or signals provided by TME cell components. Finally, due to persistent antigen stimulation they receive via tumor cells, CAR T cells become “exhausted” or dysfunctional losing their effector function and failing to evolve into effector memory T cells.

Macrophages are antigen-presenting cells that can stimulate T cells locally and thus promote adaptive anti-tumor responses. Macrophages produce proteases that can dramatically modify the extracellular matrix within the tumor mass and hence the architecture of the tumor tissue. Macrophages also have anti-tumor capabilities, such as the ability to phagocyte entire tumor cells or undertake antibody-dependent cell phagocytosis. The intrinsic features of macrophages make them an ideal candidate to overcome the limitations of CAR T cells.

Various strategies have been adopted to harness the anti-tumor capacity of myeloid cells by genetic engineering. One of the most promising strategies was to virally transduce macrophages with CAR constructs to mobilize their capacity to phagocytose tumor cells. For example in WO2017/019848, macrophages were transduced with an adenoviral vector encoding a CAR construct composed of an anti-HER2 scFv fused with a transmembrane domain and the CD3z intracellular domain. In vitro, these CAR macrophages were able to phagocyte SKOV3 cells, a HER2-expressing cell line, specifically. In vivo, they inhibited SKOV3 tumor growth, increased mouse survival, and reduced lung metastatic burden upon injection in tumor-bearing NSG (NOD/SCID/Gchainnull) mice. Adenoviral vector transduction of macrophages enhanced interferon-associated genes expression and polarized the macrophages towards an inflammatory phenotype.

However, there is a need for therapeutic cells which would be able not only to bind a given antigen, activating thus their great capacity to phagocytose tumor cells, but also to further mobilize the antigen presentation and co-stimulatory capacities of macrophages upon their encounter of tumor cells, and thus to stimulate anti-tumor immunity.

The present invention solves this need.

### SUMMARY OF THE INVENTION

The present invention thus relates to a modified cell comprising a chimeric antigen receptor (CAR), wherein said CAR comprises:
an extracellular antigen-binding domain which binds to a tumor antigen or an antigen present on cells of the TME;
a transmembrane domain; and
an intracellular signaling domain comprising the CD40 cytotail;
and wherein said modified cell is a myeloid cell.

Said myeloid cell modified by a CAR is called “CAR myeloid cell” in the present invention.

Preferably the CAR comprises a hinge domain between the extracellular antigen-binding domain and the transmembrane domain.

The present invention also relates to a modified induced pluripotent stem cell (iPS) or hematopoietic stem cell (HSC) comprising a chimeric antigen receptor (CAR), wherein said CAR comprises:
an extracellular antigen-binding domain which binds to a tumor antigen or an antigen present on cells of the TME;
optionally a hinge domain;
a transmembrane domain; and
an intracellular signaling domain comprising the CD40 cytotail.

Said iPS or HSC modified by a CAR is called “CAR iPS” or “CAR HSC”, respectively, in the present invention.

The present invention also relates to a pharmaceutical composition comprising a modified CAR myeloid cell, or a modified CAR iPS or CAR HSC, and a pharmaceutical acceptable carrier.

The present invention also relates to the use of a modified CAR myeloid cell, or a modified CAR iPS or CAR HSC, in the treatment of cancer or an inflammatory disease.

It also relates to products containing a modified CAR myeloid cell, a modified CAR iPS or a CAR HSC, and a CAR-T cell, as a combined preparation for simultaneous, separate or sequential use in treatment of cancer or an inflammatory disease.

It also relates to products containing a modified CAR myeloid cell, a modified CAR iPS or a CAR HSC, and an Immune Checkpoint Inhibitor (ICI) as a combined preparation for simultaneous, separate or sequential use in treatment of cancer or an inflammatory disease.

It further relates to a method for manufacturing a modified CAR myeloid cell, a modified CAR iPS or a CAR HSC, the method comprising:
providing at least one cell chosen from isolated myeloid cells, induced pluripotent stem cells (iPS) and hematopoietic stem cells (HSC);
transducing said cell with a vector comprising a nucleic sequence coding for said CAR, preferably a lentiviral vector.

### DETAILED DESCRIPTION OF THE INVENTION

The inventors have now synthetized novel CAR myeloid cells, which comprise the cytosolic domain of the CD40 molecule. CD40 is expressed by all macrophages and their progenitors, and enhances macrophages pro-inflammatory functions upon interaction with its ligand (CD40L), which is expressed by activated T lymphocytes. CD40 signaling triggers the secretion of pro-inflammatory cytokines, chemokines, and the expression of inducible nitric oxide synthase (iNOS) and matrix metalloproteinases.

Surprisingly, as shown in the examples, the inventors demonstrated that the CD40 cytosolic domain in the CAR constructs of the invention enhances the antigen-dependent immune response of the resulting CAR macrophages through the secretion of pro-inflammatory cytokines. The CAR according to the invention provides an efficient tumor growth control. CAR macrophages containing the CD40 domain of the invention are able to control the growth of established tumor spheroids (3D), but not of co-culture assays where macrophages and tumor cells are added at the same time or in classical 2D co-culture assays. This uncovers an unexpected feature of CAR macrophages containing the CD40 domain of the invention. As shown in the examples, CAR macrophages of the invention are indeed able to significantly control growth of established tumor spheroids (3D), which are much more representative of the clinical/in vivo situation than classical 2D co-culture or artificial 3D co-culture (see especially FIGS. 2 to 4).

No prior art document discloses the selection and the introduction of only the CD40 domain of the invention, alone in a CAR myeloid cell can unexpectedly inhibit and/or control tumor growth.

The present invention thus relates to a modified cell comprising a CAR, wherein said CAR comprises:
an extracellular antigen-binding domain which binds to a tumor antigen or an antigen present on cells of the TME;
optionally a hinge domain;
a transmembrane domain; and
an intracellular signaling domain comprising the CD40 cytotail;
and wherein said modified cell is a myeloid cell.

Said myeloid cell modified by a CAR is called “CAR myeloid cell” in the present invention.

The present invention also relates to a modified induced pluripotent stem cell (iPS) or hematopoietic stem cell (HSC) comprising a chimeric antigen receptor (CAR), wherein said CAR comprises:
an extracellular antigen-binding domain which binds to a tumor antigen or an antigen present on cells of the TME;
optionally a hinge domain;
a transmembrane domain; and
an intracellular signaling domain comprising the CD40 cytotail.

Said iPS or HSC modified by a CAR is called “CAR iPS” or “CAR HSC”, respectively, in the present invention.

### Myeloid Cell

The myeloid cell of the invention is any type of cells derived from the myeloid tissue (bone marrow), or resembling bone marrow.

Preferably, it is a monocyte, a macrophage or a dendritic cell, more preferably a monocyte.

Said myeloid cell is modified in that it expresses a CAR.

Induced Pluripotent Stem Cell (iPS) or Hematopoietic Stem Cell (HSC)

The term “stem cell” refers to a cell that, by successive divisions can give rise to specialized cells. The term “pluripotent stem cell” refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent stem cells can give rise to any fetal or adult cell type but they cannot give rise to an entire organism. A “pluripotent stem cell” may be identified by the expression of one or more of the cell markers Klf4, Sox2, Oct4, cMyc, Nanog and SSEA1. A cell is considered as a pluripotent stem cell when it is capable of generating cells from any of the three germ layers: endoderm, identified by the expression of alpha-fetoprotein; mesoderm (identified by the expression of desmin and/or alpha smooth muscle actin) and ectoderm (identified by the expression of beta-tubulin III=Tuj1 and/or E to N-cadherin). Assays to assess the pluripotentiality of a cell are known in the art.

The term “induced pluripotent stem cell” or “iPS” refers to a pluripotent cell artificially derived from a non-pluripotent cell, typically an adult somatic cell, by inducing a forced expression of certain genes. An “induced pluripotent stem cell” is defined by the expression of several transcription factors including one or more of Klf4, Sox2, Oct4 and cMyc. iPS cells are typically derived by transfection of certain stem cell-associated genes into non-pluripotent cells, such as adult fibroblasts. Transfection is typically achieved through viral vectors, such as retroviruses, and transfected genes include Oct-3/4 (Pou5fl) and Sox2. Additional genes include certain members of the Klf family (Klfl, Klf2, Klf4 and Klf5), the Myc family (c-myc, L-myc, N-myc), Nanog and LIN28 have been identified to increase the induction efficiency. After 3-4 weeks, small numbers of transfected cells begin to become morphologically and biochemically similar to pluripotent stem cells, and are typically isolated through morphological selection, doubling time, or through a reporter gene and antibiotic selection. Protocols for iPS culture are disclosed in Mochiduki and Okita, 2012. Non-pluripotent cells that can be used to obtain iPS are, without limitation, fibroblasts, keratinocytes and adipocytes. These cells can be obtained from an adult being by methods well-known in the state of the art (Mochiduki and Okita, 2012).

The «hematopoietic stem cells» (HSC) possess the ability to fully reconstitute the immune system of a lethally irradiated host from which the cells are obtained. The hematopoietic stem cells give rise to all blood and immune cells.

Said iPS or HSC is modified in that it expresses a CAR.

The CAR myeloid cell, the CAR iPS or CAR HSC comprise a CAR, which is detailed below.

### Chimeric Antigen Receptor (CAR)

The CAR of the invention comprises, from its N-terminal end to its C-terminal end:
an extracellular antigen-binding domain which binds to a tumor antigen or an antigen present on cells of the TME;
optionally a hinge domain;
a transmembrane domain; and
an intracellular signaling domain comprising the CD40 cytotail.

Between each domain, a linker, identical or different, may be present. Preferably, the CAR does not comprise any linker between the different domains. In other words, the CAR is obtained by direct fusion of the different domains.

### Antigen-Binding Domain

The CAR myeloid cell according to the invention, or the CAR iPS or CAR HSC according to the invention, comprises, at the N-terminal end of the CAR, an extracellular antigen-binding domain which binds to a tumor antigen or an antigen present on cells of the TME (i.e. a TME antigen).

The “antigen-binding domain” may be any polypeptide or fragment thereof, such as an antibody fragment variable domain, either naturally-derived or synthetic, which binds to an antigen. Antigen-binding domains notably include polypeptides derived from antibodies, such as single chain variable fragments (scFv), Fab, Fab′, F(ab′)2, Fv fragments and nanobodies; polypeptides derived from T cell receptors (TCR), such as TCR variable domains; and any ligand or receptor fragment that binds to the antigen. Said antigen-binding domain has antigen specificity for a tumor antigen or a TME antigen. An «antigen-binding domain which has antigen specificity for a tumor antigen” is an antigen-binding domain that binds to an antigen on a tumor. An «antigen-binding domain which has antigen specificity for a TME antigen” is an antigen-binding domain that binds to an antigen which is present on cells of the tumor microenvironment (TME). The TME includes the tissues and cells around a tumor; it notably includes the surrounding blood vessels, immune cells such as Treg cells or immunosuppressive macrophages, fibroblasts, signaling molecules and the extracellular matrix.

Preferably, the tumor antigen is chosen from antigens expressed at the surface of tumor cells at higher levels than on other cell types. Preferably, the tumor antigen is chosen from CD19, MUC16, MUC1, CA1X, carcinoembryonic antigen (CEA), CD8, CD7, CD 10, CD20, CD22, CD30, CLL1, CD33, CD34, CD38, CD41, CD44, CD49f, CD56, CD74, CD133, CD138, EGP-2, EGP-40, EpCAM, erb-B2,3,4, FBP, Fetal acetylcholine receptor, folate receptor-a, GD2, GD2Ac, GD3, ITER-2, hTERT, IL-I3R-a2, K-light chain, KDR, LeY, LI cell adhesion molecule, MAGE-A1, Mesothelin, ERBB2, MAGEA3, p53, MARTI, GPI00, Proteinase 3 (PR1), Tyrosinase, Survivin, EphA2, NKG2D ligands, NY-ES0-1, oncofetal antigen (h5T4), PSCA, PSMA, ROR1, TAG-72, VEGF-R2, WT-I, BCMA, CD123, CD44V6, NKCS1, EGF1R, EGFR-VIII, CD99, CD70, ADGRE2, CCR1, LILRB2, PRAME, CCR4, CD5, CD3, TRBC1, TRBC2, TIM-3, Integrin B7, ICAM-I, CD70, Tim3, CLEC12A, ER, human telomerase reverse transcriptase (hTERT), mouse double minute 2 homolog (MDM2), cytochrome P450 1B1 (CYP1B), HER2/neu, p95HER2, Wilms' tumor gene 1 (WT1), livin, alphafetoprotein (AFP), prostate-specific membrane antigen (PSMA), cyclin (DI), mesothelin, B-cell maturation antigen (BCMA) and tumor-associated calcium signal transducer 2 (TROP2).

Preferably, the TME antigen is chosen from antigens expressed by activated CAF such as FAP, antigens expressed by T regs and antigens expressed by protumoral myeloid cells such as TREM-2. Preferably, the TME antigen is chosen from FAP, antigens expressed by T regs and TREM-2.

Preferably, the tumor antigen or TME antigen is CD19. More preferably, the extracellular antigen-binding domain which binds to a tumor antigen or a TME antigen is an anti-CD19 binding domain, preferably an anti-CD19 scFV.

Preferably, the extracellular antigen-binding domain comprises the amino acid sequence:

(SEQ ID NO: 1)
MALPVTALLLPLALLLHAARPDIQMTQTTSSLSASLGDRVTISCR
ASQDISKYLNWYQQKPDGTVKLLIYHTSRLHSGVPSRFSGSGSGT
DYSLTISNLEQEDIATYFCQQGNTLPYTFGGGTKLEITGGGGSGG
GGSGGGGSEVKLQESGPGLVAPSQSLSVTCTVSGVSLPDYGVSWI
RQPPRKGLEWLGVIWGSETTYYNSALKSRLTIIKDNSKSQVFLKM
NSLQTDDTAIYYCAKHYYYGGSYAMDYWGQGTSVTVSS.

### Hinge Domain

The CAR may comprise a hinge domain. Said hinge domain confers flexibility to the CAR obtained.

The hinge domain may be any hinge domain present in immunoglobulins or in CD molecules.

Preferably, the hinge domain is the one of CD8. CD8 comprises an alpha chain (CD8a) and a beta chain (CD8b). Preferably, the hinge domain is the one of the CD8a chain.

The human version of CD8a may be found in Uniprot under accession number Q8TAW8. CD8a comprises 235 amino acids. The hinge domain is the fragment of amino acids 138 to 182 of said sequence, which corresponds to SEQ ID NO:2.

Preferably, the hinge domain is the one of CD8a, preferably of human CD8a.

Preferably, the hinge domain comprises the amino acid sequence TTTPAPRPPTPAPTIASQPLSLRPEACRPAAGGAVHTRGLDFACD (SEQ ID NO:2).

### Transmembrane Domain

The CAR comprises a transmembrane domain. Said transmembrane domain may be a single-pass or a multipass transmembrane sequence.

Single-pass transmembrane regions are found in certain CD molecules, tyrosine kinase receptors, serine/threonine kinase receptors, TGF, BMP, activin and phosphatases. Single-pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane.

Multipass transmembrane domains are present in proteins such as ion pumps, ion channels and transporters, and include two or more helices that span the membrane multiple times.

Sequences for single-pass and multipass transmembrane domains are known and can be selected for incorporation into the CAR.

The transmembrane domain can be chosen from wild-type transmembrane domains and mutated transmembrane domains. Mutated transmembrane domains may be modified by a mutation, such as an amino acid substitution (for example, an amino acid which is typically charged is substituted by a hydrophobic residue). Preferably, the transmembrane domain is the one of the alpha, beta or zeta chain of the T cell receptor, CD3-8, CD3zeta, CD4, CD5, CD8, CD8a, CD9, CD16, CD22, CD28, CD33, CD38, CD64, CD80, CD86, CD134, CD137 or CD154. Preferably, the transmembrane domain is a CD8 transmembrane domain.

The transmembrane domain may also be synthesized de novo, comprising mostly hydrophobic residues, such as, for example, leucine and valine.

According to the invention, the transmembrane domain is fused at its N-terminal end to the extracellular antigen-binding domain of the CAR, and at its C-terminal end to the intracellular signaling domain.

In certain embodiments, a short polypeptide linker may form the linkage between the transmembrane domain and the intracellular signaling domain of the CAR.

The CAR may further comprise a stalk, that is, an extracellular region of amino acids between the extracellular antigen-binding domain and the transmembrane domain. For example, the stalk may be a sequence of amino acids naturally associated with the selected transmembrane domain.

Preferably, the CAR comprises a CD8 transmembrane domain. Preferably, the CAR comprises a CD8 transmembrane domain, and a CD8 hinge domain. Said hinge domain is preferably fused (preferably directly), at its C-terminal end, to the N-terminal end of the transmembrane domain.

Preferably, the transmembrane domain comprises the amino acid sequence IYIWAPLAGTCGVLLLSLVITLYC (SEQ ID NO:3). This transmembrane domain is the one of human CD8.

Preferably, the hinge domain comprises the amino acid sequence TTTPAPRPPTPAPTIASQPLSLRPEACRPAAGGAVHTRGLDFACD (SEQ ID NO:2).

### Intracellular Signaling Domain

The CAR myeloid cell according to the invention, or the CAR iPS or CAR HSC according to the invention, comprises an intracellular signaling domain at the C-terminal end of the CAR.

Specifically, said intracellular signaling domain comprises the CD40 cytoplasmic tail (or cytotail).

Preferably, said intracellular signaling domain consists of the CD40 cytoplasmic tail.

By “CD40 cytotail”, it is meant the cytosolic domain of the CD40 molecule. CD40, also called TNFRSF5, is a costimulatory protein found on antigen-presenting cells, and is required for their activation. The sequence of human CD40 (hCD40) may be found in Uniprot under accession number P25942. It comprises 277 amino acids. The fragment comprising amino acids 216-277 of said sequence is the cytosolic part. Said fragment corresponds to SEQ ID NO:4.

Preferably, the intracellular signaling domain comprises a CD40 cytotail which is a fragment of human CD40.

Preferably, the intracellular signaling domain comprises the amino acid sequence KKVAKKPTNKAPHPKQEPQEINFPDDLPGSNTAAPVQETLHGCQPVTQEDGKESRISVQ ERQ (SEQ ID NO:4).

Preferably, the intracellular signaling domain only consists in a CD40 cytotail which is a fragment of human CD40.

Preferably, the intracellular signaling domain only consists in the amino acid sequence KKVAKKPTNKAPHPKQEPQEINFPDDLPGSNTAAPVQETLHGCQPVTQEDGKESRISVQ ERQ (SEQ ID NO:4).

Preferably, the CAR comprises, from its N-terminal end to its C-terminal end:
an extracellular antigen-binding domain of sequence SEQ ID NO:1,
optionally a hinge domain of sequence SEQ ID NO:2,
a transmembrane domain of sequence SEQ ID NO:3,
an intracellular signaling domain of sequence SEQ ID NO:4.

The CAR myeloid cell according to the invention, or the CAR iPS or CAR HSC according to the invention, preferably presents targeted effector activity. By “targeted effector activity”, it is meant at least one effector activity chosen from phagocytosis, targeted cellular cytotoxicity, production of cytokines, production of reactive oxygen species (ROS), myeloid activation and antigen processing and presentation to T cells. Preferably, the targeted effector activity is selected from antigen-dependent phagocytosis of tumor cells and antigen-dependent tumor cell cytokine secretion. Antigen-dependent phagocytosis of tumor cells and antigen-dependent tumor cell cytokine secretion may be measured according to methods well-known in the art, which are illustrated in the examples.

The targeted effector activity of the CAR myeloid cell according to the invention may be measured by using an in vitro assay method for obtaining 3D spheroids.

Said method allows obtaining 3D spheroids, which are a much better reliable model of in vivo expectations than 2D co-culture systems. Said in vitro method typically comprises:
a) Culturing at least one tumor cell line or at least one tumor cell from a primary tumor, and optionally at least one myeloid cell line, in ultra-low binding plate, so that all cell lines growth in a spheroid form;
b) Following the growth of the 3D spheroid (co-)cultured cell lines by time-lapse microscopy;
c) Optionally, analyzing a sample of the 3D spheroid (co-)cultured cell lines and/or the supernatant. This step can be performed to analyze the supernatant composition and/or by 3D imaging. For example, this step may comprise time-lapse microscopy (3D imaging) or supernatant chemical analysis.

Preferably, said in vitro method is for assessing the targeted effector activity of the CAR myeloid cell according to the invention (preferably antigen-dependent phagocytosis of tumor cells) and comprises:
a) Culturing at least one tumor cell line or at least one tumor cell from a primary tumor in ultra-low binding plate, so that all cell lines or cells growth in a spheroid form;
b) Following the growth of the spheroid cultured cell lines or cells by time-lapse microscopy, in order to obtain at least one 3D spheroid;
c) Adding at least one CAR myeloid cell according to the invention to the 3D spheroid obtained in step b), to obtain a 3D spheroid culture; and
d) Analyzing a sample of the 3D spheroid culture obtained in step c) and/or the supernatant thereof, for example by time-lapse microscopy or supernatant chemical analysis.

In any case, the ultra-low binding plate of step a) preferably is non-adherent. Preferably, it comprises no matrix or other solid support, and comprises a liquid medium. Preferably, it is a U-shape plate. With such a shape, the cells typically adhere together in the liquid medium and form a single spheroid in each well.

The tumor cell line may be any tumor cell line known in the art, such as A549 or MDA-MB-231.

The tumor cell may also come from a primary tumor. By primary tumor, it is meant the original, or first, tumor in the body. Cancer cells from a primary tumor may spread to other parts of the body and form new (or secondary) tumors; this is called metastasis.

Said in vitro method is illustrated in the examples below.

The CAR myeloid cell according to the invention, or the CAR iPS or CAR HSC according to the invention, preferably comprises an additional vector, said vector comprising a sequence coding for a gene of interest under the control of a cytokine specific promoter.

Preferably, the gene of interest is chosen from the genes coding for IFNgamma, the genes coding for IFNalpha, the genes coding for IFNbeta, the genes coding for IFNlambda, the genes coding for IL12 and the genes coding for IL10 or TGFbeta.

Preferably, the gene of interest is a human gene.

The present invention also relates to the nucleic acid sequence coding for said CAR. Said nucleic acid sequence may be a DNA or RNA sequence. Said nucleic acid sequence may be used in therapy, especially for treating a cancer or an inflammatory disease. Preferably, said nucleic acid sequence is administered to a subject, preferably by injection. Accordingly, the macrophages of said subject receive said nucleic acid sequence, and subsequently express the CAR.

Preferably, the nucleic sequence coding for the intracellular domain of said CAR is the sequence SEQ ID NO:5.

### Therapeutic Uses

The present invention also relates to the use of a CAR myeloid cell according to the invention, or a CAR iPS or CAR HSC according to the invention, as a medicament.

The present invention also relates to a pharmaceutical composition comprising a CAR myeloid cell according to the invention, or a CAR iPS or CAR HSC according to the invention, and a pharmaceutical acceptable carrier.

The present invention also relates to the use of a CAR myeloid cell according to the invention, or a CAR iPS or CAR HSC according to the invention, or of the pharmaceutical composition described above, in the treatment of cancers or an inflammatory disease. The inflammatory disease may be an autoimmune disease.

The present invention also relates to products containing a CAR myeloid cell according to the invention, or a CAR iPS or CAR HSC according to the invention, and a CAR-T cell, as a combined preparation for simultaneous, separate or sequential use in treatment of cancer or an inflammatory disease.

CAR-T cells are well-known in the art. Preferably, CAR-T cells are chosen from tisagenlecleucel, axicabtagene ciloleucel, brexucabtagene autoleucel, lisocabtagene maraleucel and idecabtagene vicleucel.

The present invention also relates to products containing a CAR myeloid cell according to the invention, or a CAR iPS or CAR HSC according to the invention, and an Immune Checkpoint Inhibitor (ICI) as a combined preparation for simultaneous, separate or sequential use in treatment of cancer or an inflammatory disease.

An “immune checkpoint inhibitor” refers to any compound inhibiting the function of an immune checkpoint protein. Inhibition includes reduction of function and full blockade. In particular, the immune checkpoint protein is a human immune checkpoint protein. Thus the immune checkpoint protein inhibitor is preferably an inhibitor of a human immune checkpoint protein.

Immune checkpoint proteins that may be quoted are CTLA-4, PD-1, PD-L1, PD-L2, LAG-3, BTLA, B7H3, B7H4, TIM3, KIR (such as KIR3DL2, KIR2DL1/2/3, KIR2L3), TIGIT, VISTA, IDO, CEACAM-1 or A2aR.

The immune checkpoint inhibitors may be drugs such as small molecules, recombinant forms of ligand or receptors, or preferably antibodies, such as human antibodies. Known inhibitors of the immune checkpoint proteins or analogs thereof may be used, in particular chimerized, humanized or human forms of antibodies.

Preferably the ICI is selected from an inhibitor of CTLA-4, PD-1, PD-L1, PD-L2, LAG-3, BTLA, B7H3, B7H4, TIM3, KIR (such as KIR3DL2, KIR2DL1/2/3, KIR2L3), TIGIT, VISTA, IDO, CEACAM-1 or A2aR. Preferably, the ICI is an anti-CTLA-4 antibody, more preferably tremelimumab or ipilimumab. In certain aspects, the ICI is an anti-killer-cell immunoglobulin-like receptor (KIR) antibody, more preferably lirilumab and IPH4102. Preferably, the ICI is an anti-PD-1 antibody, more preferably chosen from nivolumab (ONO-4538, BMS-936558, MDX1106, GTPL7335 or Opdivo), pembrolizumab (MK-3475, MK03475, lambrolizumab, SCH-900475 or Keytruda), pidilizumab, AMP-514, cemiplimab (REGN2810), CT-011, BMS 936559, MPDL3280A, AMP-224, tislelizumab (BGB-A317), spartalizumab (PDR001 or PDR-001), ABBV-181, JNJ-63723283, BI 754091, MAG012, TSR-042, AGEN2034 and antibodies described in International patent applications WO2004004771, WO2004056875, WO2006121168, WO2008156712, WO2009014708, WO2009114335, WO2013043569 and WO2014047350. Preferably, the inhibitor of PD-L1 is durvalumab, atezolizumab, LY3300054 or avelumab. Preferably, the inhibitor of PD-L2 is rHIgM12B7. Preferably, the LAG3 inhibitor is IMP321, BMS-986016 or inhibitors of the LAG3 receptor described in U.S. Pat. No. 5,773,578. Preferably, the inhibitor of A2aR is PBF-509. Preferably, the inhibitor of CTLA-4 is an anti-CTLA-4 antibodies including, but not limited to, ipilimumab (see, e.g., U.S. Pat. Nos. 6,984,720 and 8,017,114), tremelimumab (see, e.g., U.S. Pat. Nos. 7,109,003 and 8,143,379), single chain anti-CTLA4 antibodies (see, e.g., International patent applications WO1997020574 and WO2007123737) and antibodies described in U.S. Pat. No. 8,491,895. Example of anti-VISTA antibodies are described in US patent application US20130177557. Preferably, the ICI is chosen from tremelimumab, ipilimumab, lirilumab, nivolumab, pembrolizumab, pidilizumab, AMP-514, REGN2810, CT-011, BMS 936559, MPDL3280A, AMP-224, durvalumab, atezolizumab, avelumab, rHIgM12B7, IMP321, BMS-986016 and PBF-509.

The present invention also relates to products containing a CAR myeloid cell according to the invention, or a CAR iPS or CAR HSC according to the invention, and an immune checkpoint therapy related to co-stimulatory antibodies delivering positive signals through immune-regulatory receptors including but not limited to ICOS, CD137, CD27, OX-40 and GITR as a combined preparation for simultaneous, separate or sequential use in treatment of cancer or an inflammatory disease.

The present invention also relates to products containing a CAR myeloid cell according to the invention, or a CAR iPS or CAR HSC according to the invention, and additional cancer therapies as a combined preparation for simultaneous, separate or sequential use in treatment of cancer. In particular, products containing a CAR myeloid cell according to the invention, or a CAR iPS or CAR HSC according to the invention may be administered in combination with targeted therapy, immunotherapy such as immune checkpoint therapy and/or immune checkpoint inhibitor, co-stimulatory antibodies, chemotherapy and/or radiotherapy.

In some embodiments, the products containing a CAR myeloid cell according to the invention, or a CAR iPS or CAR HSC according to the invention may be used in combination with targeted therapy. As used herein, the term “targeted therapy” refers to targeted therapy agents, drugs designed to interfere with specific molecules necessary for tumor growth and progression. For example, targeted therapy agents such as therapeutic monoclonal antibodies target specific antigens found on the cell surface, such as transmembrane receptors or extracellular growth factors. Small molecules can penetrate the cell membrane to interact with targets inside a cell. Small molecules are usually designed to interfere with the enzymatic activity of the target protein such as for example proteasome inhibitor, tyrosine kinase or cyclin-dependent kinase inhibitor, histone deacetylase inhibitor. Targeted therapy may also use cytokines. Examples of such targeted therapy include: Ado-trastuzumab emtansine (HER2), Afatinib (EGFR (HER1/ERBB1), HER2), Aldesleukin (Proleukin), alectinib (ALK), Alemtuzumab (CD52), axitinib (kit, PDGFRbeta, VEGFR1/2/3), Belimumab (BAFF), Belinostat (HDAC), Bevacizumab (VEGF ligand), Blinatumomab (CD19/CD3), bortezomib (proteasome), Brentuximab vedotin (CD30), bosutinib (ABL), brigatinib (ALK), cabozantinib (FLT3, KIT, MET, RET, VEGFR2), Canakinumab (IL-1 beta), carfilzomib (proteasome), ceritinib (ALK), Cetuximab (EGFR), cofimetinib (MEK), Crizotinib (ALK, MET, ROS1), Dabrafenib (BRAF), Daratumumab (CD38), Dasatinib (ABL), Denosumab (RANKL), Dinutuximab (B4GALNT1 (GD2)), Elotuzumab (SLAMF7), Enasidenib (IDH2), Erlotinib (EGFR), Everolimus (mTOR), Gefitinib (EGFR), Ibritumomab tiuxetan (CD20), Sonidegib (Smoothened), Sipuleucel-T, Siltuximab (IL-6), Sorafenib (VEGFR, PDGFR, KIT, RAF), (Tocilizumab (IL-6R), Temsirolimus (mTOR), Tofacitinib (JAK3), Trametinib (MEK), Tositumomab (CD20), Trastuzumab (HER2), Vandetanib (EGFR), Vemurafenib (BRAF), Venetoclax (BCL2), Vismodegib (PTCH, Smoothened), Vorinostat (HDAC), Ziv-aflibercept (PIGF, VEGFA/B), Olaparib (PARP inhibitor).

In some embodiments, the products containing a CAR myeloid cell according to the invention, or a CAR iPS or CAR HSC according to the invention may be used in combination with chemotherapy. As used herein, the term “antitumor chemotherapy” or “chemotherapy” has its general meaning in the art and refers to a cancer therapeutic treatment using chemical or biochemical substances, in particular using one or several antineoplastic agents or chemotherapeutic agents. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin as such hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; and pharmaceutically acceptable salts, acids or derivatives of any of the above.

In some embodiments, the products containing a CAR myeloid cell according to the invention, or a CAR iPS or CAR HSC according to the invention is administered to the patient in combination with radiotherapy for simultaneous, separate or sequential use in treatment of cancer or an inflammatory disease. Suitable examples of radiation therapies include external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8(1):16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin M C. The radiotherapy FLASH to save healthy tissues. Med Sci (Paris) 2015; 31:121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11).

It is also described a method for treating cancer or an inflammatory disease in a subject in need thereof, comprising a step of administering to said subject a therapeutically effective amount of a CAR myeloid cell according to the invention, or a CAR iPS or CAR HSC according to the invention. It is also described a method for treating cancer or an inflammatory disease, which comprises:
collecting myeloid cells from a patient;
modifying at least one of said myeloid cells by transducing said cell with a vector comprising a nucleic sequence coding for the CAR, preferably a lentiviral vector; and
re-injecting said modified myeloid cells into the patient.

Cancer refers to tumors. The tumors to be treated include primary tumors and metastatic tumors, as well as refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents, but recur up to five years, sometimes up to ten years or longer after treatment is discontinued.

Examples of cancers that may be treated by the CAR myeloid cell according to the invention, or the CAR iPS or CAR HSC according to the invention, include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/ squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extramammary paraganglioma, malignant; pheochromocytoma; glomangio sarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Preferably, cancer is a solid tumor or a metastasis.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment.

By “therapeutic regimen” is meant the pattern of treatment of an illness, e.g., the pattern of dosing used during therapy. A therapeutic regimen may include an induction regimen and a maintenance regimen.

The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both.

The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

By a “therapeutically effective amount”, it is meant a sufficient amount of a CAR myeloid cell according to the invention, or a CAR iPS or CAR HSC according to the invention, to treat the disease (e.g. cancer) at a reasonable benefit/risk ratio applicable to any medical treatment. It will be understood that the total daily usage of the product of the present invention will be decided by the attending physician within the scope of sound medical judgment. The specific therapeutically effective dose level for any particular patient will depend upon a variety of factors including the age, body weight, general health, sex and diet of the patient; the time of administration, route of administration, and rate of excretion of the specific compound employed; the duration of the treatment; drugs used in combination or coincidental with the product; and like factors well known in the medical arts. For example, it is well known within the skill of the art to start doses of the compound at levels lower than those required to achieve the desired therapeutic effect and to gradually increase the dosage until the desired effect is achieved.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The product can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Preparation Method

The present invention also relates to a method for manufacturing a CAR myeloid cell according to the invention, or a CAR iPS or CAR HSC according to the invention, which comprises:
providing at least one cell chosen from isolated myeloid cells, induced pluripotent stem cells (iPS) and isolated hematopoietic stem cells (HSC); and
transducing said cell with a vector comprising a nucleic sequence coding for said CAR, preferably a lentiviral vector.

Of course, said CAR comprises:
an extracellular antigen-binding domain which binds to a tumor antigen or a TME antigen;
optionally a hinge domain;
a transmembrane domain; and
an intracellular signaling domain comprising the CD40 cytotail.

The first step of the preparation method is the provision of at least one cell chosen from isolated myeloid cells, induced pluripotent stem cells (iPS) and isolated hematopoietic stem cells (HSC).

Then, said cells are transduced with a vector comprising a nucleic sequence coding for said CAR, preferably a lentiviral vector. The vector may be used to introduce the CAR into an isolated myeloid cell, an iPS or an isolated HSC, preferably a monocyte, macrophage or dendritic cell. Said vector comprises a nucleic acid sequence encoding the CAR of the invention. In one embodiment, the vector is a plasmid vector, a viral vector, a retrotransposon (e.g. piggyback, sleeping beauty) or a site directed insertion vector (e.g. CRISPR, Zn finger nucleases, TALEN). Preferably, the vector is a viral vector, preferably a lentiviral vector. Vectors, including those derived from retroviruses such as lentivirus, are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells. They also have the added advantage of resulting in low immunogenicity in the subject into which they are introduced.

The expression of natural or synthetic nucleic acids is typically achieved by operably linking a nucleic acid to a promoter, and incorporating the construct into an expression vector. The vector is one generally capable of replication in a mammalian cell, and/or also capable of integration into the cellular genome of the mammal. Typical vectors contain transcription and translation terminators, initiation sequences and promoters useful for regulation of the expression of the desired nucleic acid sequence.

The nucleic sequence (nucleic acid) coding for said CAR can be cloned into any number of different types of vectors. For example, the nucleic acid can be cloned into a vector including, but not limited to a plasmid, a phagemid, a phage derivative, an animal virus or a cosmid. Vectors of particular interest include expression vectors, replication vectors, probe generation vectors and sequencing vectors.

The expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY). Viruses which are useful as vectors include retroviruses, adenoviruses, adeno-associated viruses, herpes viruses and lentiviruses. In general, a suitable vector contains an origin of replication functional in at least one organism, a promoter sequence, convenient restriction endonuclease sites, and one or more selectable markers. Additional promoter elements, e.g., enhancers, regulate the frequency of transcriptional initiation. Depending on the promoter, it appears that individual elements can function either cooperatively or independently to activate transcription.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, such as the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the actin promoter, the myosin promoter, the hemoglobin promoter and the creatine kinase promoter. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include a metallothionine promoter, a glucocorticoid promoter, a progesterone promoter and a tetracycline promoter.

In order to assess expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like. Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Preferably, the nucleic acid sequence coding for the intracellular domain of said CAR is the sequence SEQ ID NO:5.

The method may further comprise introducing into said cell an additional vector, said vector comprising a sequence coding for a gene of interest under the control of a cytokine specific promoter.

Preferably, the gene of interest is chosen from the genes coding for IFNgamma, the genes coding for IFNalpha, the genes coding for IFNbeta, the genes coding for IFNlambda, the genes coding for IL 12 and the genes coding for IL10 or TGFbeta.

Preferably, the gene of interest is a human gene.

The sequences of the present application are as follows:

| SEQ | | |
|---|---|---|
| ID | | |
| NO | Definition | Sequence |
| 1 | Extracellular | MALPVTALLLPLALLLHAARPDIQMTQTTS |
| | antigen- | SLSASLGDRVTISCRASQDISKYLNWYQQK |
| | binding | PDGTVKLLIYHTSRLHSGVPSRFSGSGSGT |
| | domain of CAR | DYSLTISNLEQEDIATYFCQQGNTLPYTFG |
| | of the | GGTKLEITGGGGSGGGGSGGGGSEVKLQES |
| | invention | GPGLVAPSQSLSVTCTVSGVSLPDYGVSWI |
| | (anti-CD19 | RQPPRKGLEWLGVIWGSETTYYNSALKSRL |
| | scFv) | TIIKDNSKSQVFLKMNSLQTDDTAIYYCAK |
| | | HYYYGGSYAMDYWGQGTSVTVSS |
| 2 | Hinge domain | TTTPAPRPPTPAPTIASQPLSLRPEACRPA |
| | of | AGGAVHTRGLDFACD |
| | CAR of the | |
| | invention | |
| | (fragment of | |
| | human CD8a) | |
| 3 | Transmembrane | IYIWAPLAGTCGVLLLSLVITLYC |
| | domain of CAR | |
| | of the | |
| | invention | |
| | (CD8 fragment) | |
| 4 | Intracellular | KKVAKKPTNKAPHPKQEPQEINFPDDLPGS |
| | signaling | NTAAPVQETLHGCQPVTQEDGKESRISVQE |
| | domain of CAR | RQ |
| | of the | |
| | invention | |
| | (human CD40 | |
| | fragment) | |
| 5 | Nucleic acid | Atggccttaccagtgaccgccttgctcctg |
| | coding for the | ccgctggccttgctgctccacgccgccagg |
| | intracellular | ccggacatccagAtgacacagactacatcc |
| | domain of a | tccctgtctgcctctctgggagacagagtc |
| | CAR of the | accatcagttgcagggcaagtcagGacatt |
| | invention | agtaaatatttaaattggtatcagcagaaa |
| | | ccagatggaactgttaaactcctgatctac |
| | | catacatcaAgattacactcaggagtccca |
| | | tcaaggttcagtggcagtgggtctggaaca |
| | | gattattctctcaccattagcaaCctggag |
| | | caagaagatattgccacttacttttgccaa |
| | | cagggtaatacgcttccgtacacgttcgga |
| | | ggggggAccaagctggagatcacaggtggc |
| | | ggtggctcgggcggtggtgggtcgggtggc |
| | | ggcggatctgaggtgaAactgcaggagtca |
| | | ggacctggcctggtggcgccctcacagagc |
| | | ctgtccgtcacatgcactgtctcaggggTc |
| | | tcattacccgactatggtgtaagctggatt |
| | | cgccagcctccacgaaagggtctggagtgg |
| | | ctgggagtaataTggggtagtgaaaccaca |
| | | tactataattcagctctcaaatccagactg |
| | | accatcatcaaggacaactccaagaGccaa |
| | | gttttcttaaaaatgaacagtctgcaaact |
| | | gatgacacagccatttactactgtgccaaa |
| | | cattattactacGgtggtagctatgctatg |
| | | gactactggggccaaggaacctcagtcacc |
| | | gtctcctcaaccacgacgccagcgcCgcga |
| | | ccaccaacaccggcgcccaccatcgcgtcg |
| | | cagcccctgtccctgcgcccagaggcgtgc |
| | | cggccAgcggcggggggcgcagtgcacacg |
| | | agggggctggacttcgcctgtgatatctac |
| | | atctgggcgcccttggcCgggacttgtggg |
| | | gtccttctcctgtcactggttatcaccctt |
| | | tactgcaaaaaggtggccaagaagccaacc |
| | | aaTaaggccccccaccccaagcaggaaccc |
| | | caggagatcaattttcccgacgatcttcct |
| | | ggctccaacactgctgctccagtgcaggag |
| | | actttacatggatgccaaccggtcacccag |
| | | gaggatggcaaagagagtcgcatctcagtg |
| | | caggagagacagtga |

The invention is now illustrated by the following figures and examples.

### FIGURES

The figures of the present application as the following:

FIG. 1. Lentiviral transduction of monocytes allows high surface expression of CAR constructs

A. Schematic representation of the CAR constructs cloned into a lentivector. scFv; single chain antibody specific for CD19. TM: transmembrane domain. CD40 corresponds to the intracellular domain of the molecule. A hinge domain is also present in each CAR construct, between the scFv and the TM domains, but is not illustrated.

B. Representative histograms of transduced macrophages stained for CD19 expression. Purified CD14+ cells were transduced with lentivectors encoding the indicated CAR constructs, cultured for 10 days in the presence of M-CSF but without any selection. Cells were then collected and analyzed by flow cytometry using a rhCD19-Atto647 conjugated protein for staining.

Note the high efficiency of the transduction which is routinely above 90%.

FIG. 2. CAR Macrophages can phagocytose A549-CD19⁺ cells

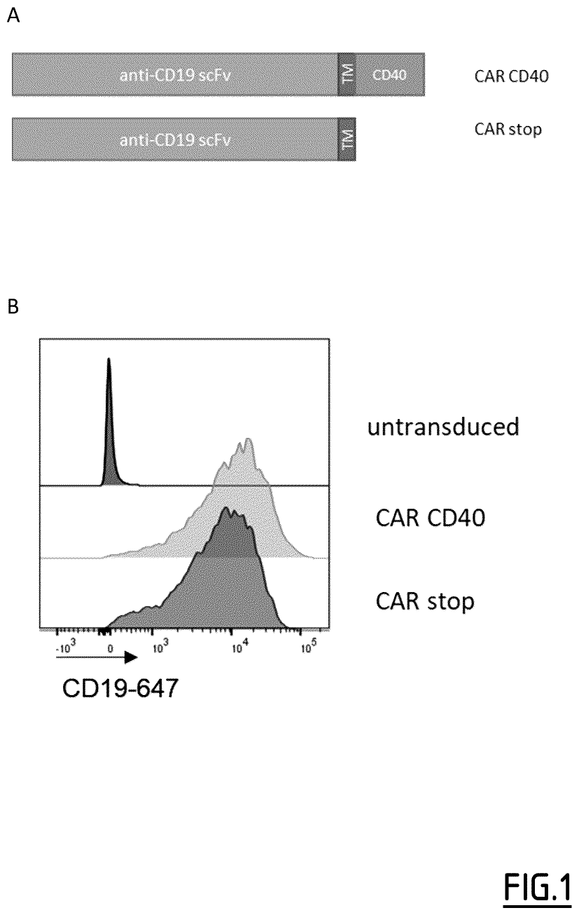

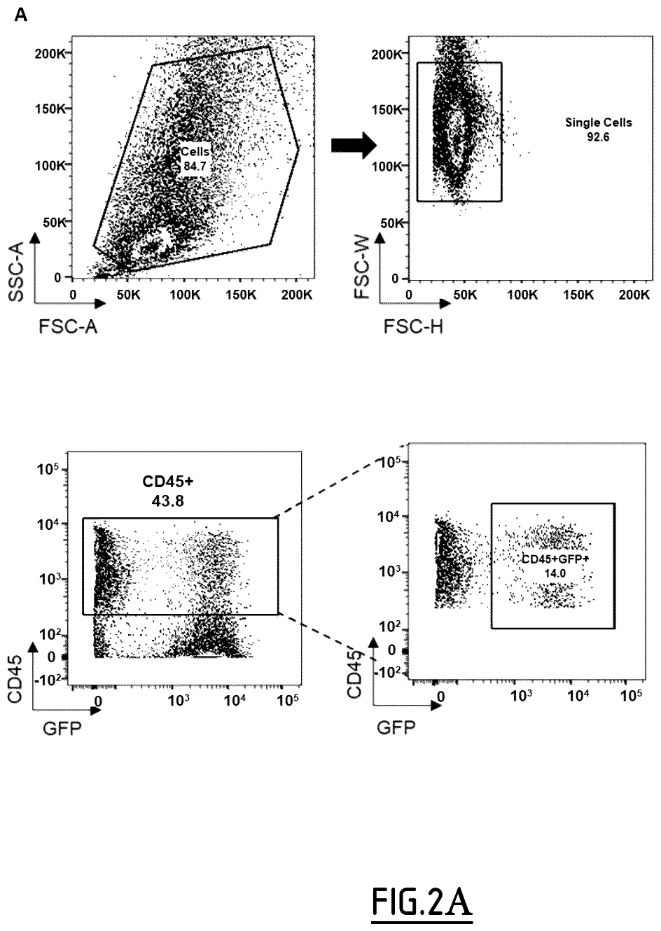

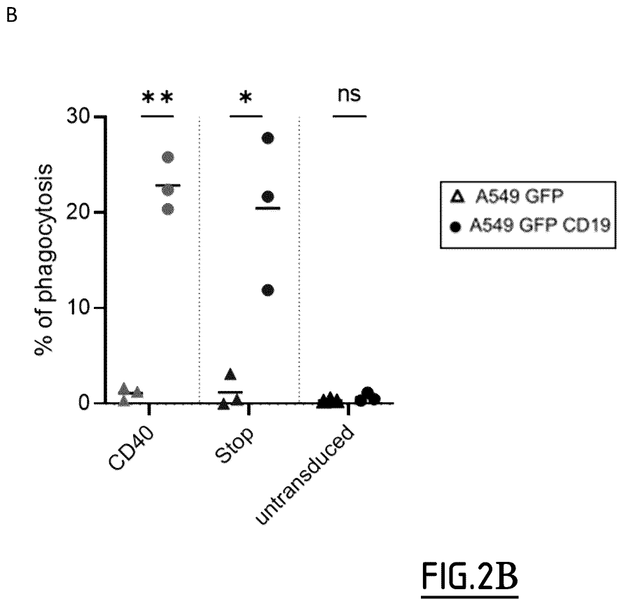

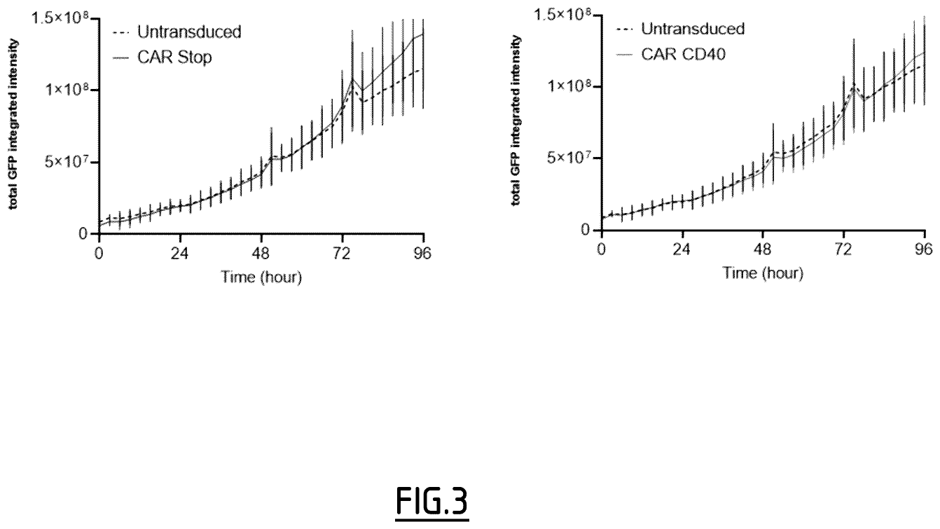

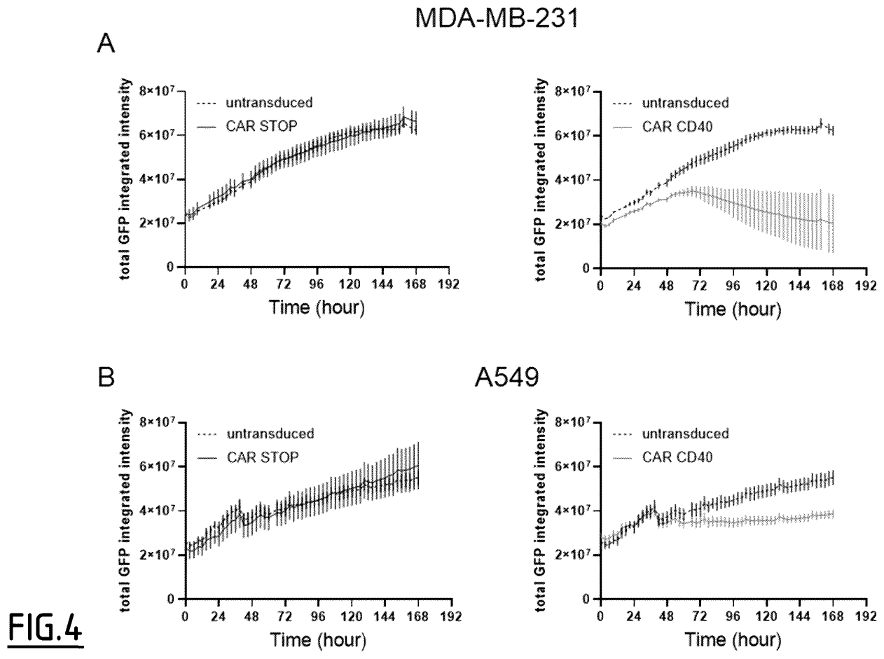

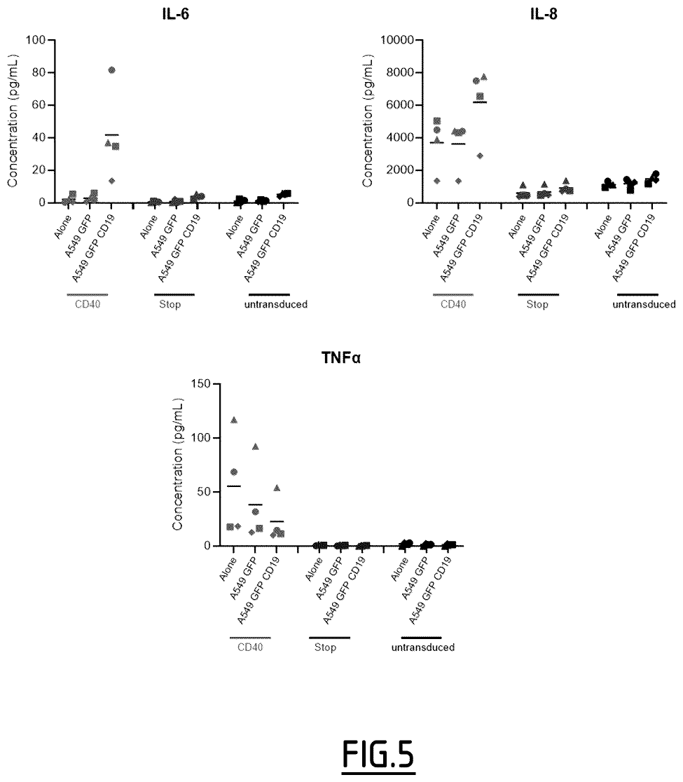

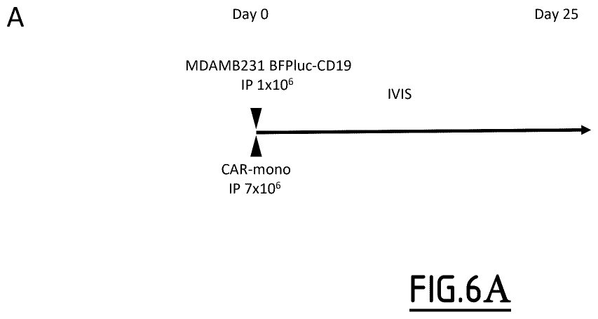

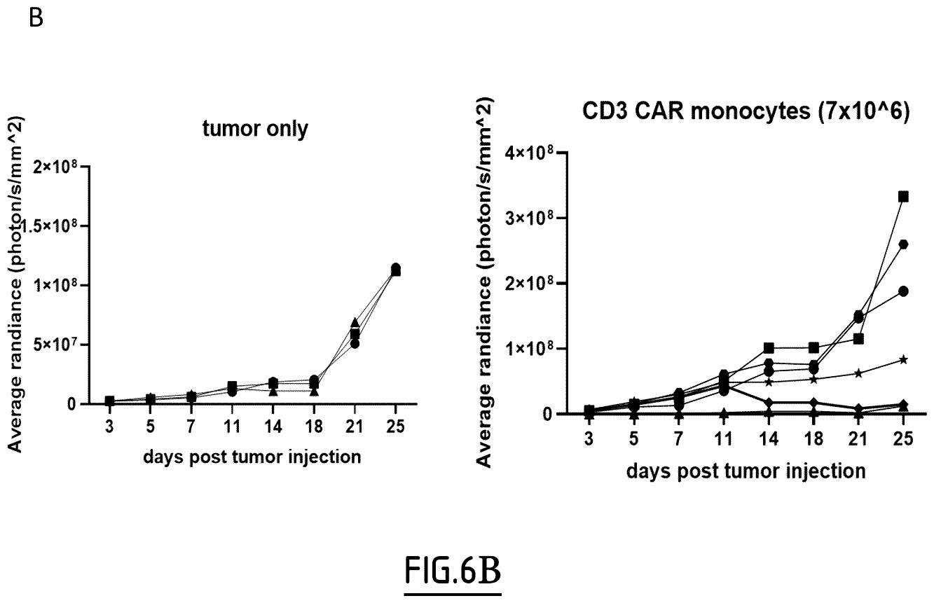

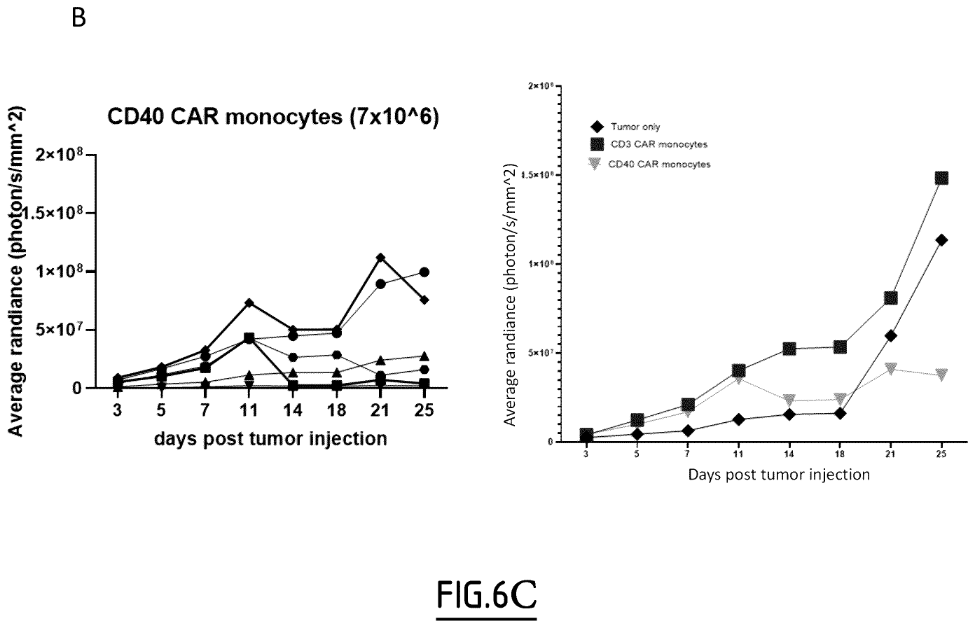

FACS-based phagocytosis assay of A549-GFP or A549-GFP-CD19⁺ by CAR Macrophages at a 1:1 ET ratio. Macrophages were prepared as for FIG. 1 and then incubated for 2 hours with A549 cells expressing or not CD19.

A. Flow cytometry gating strategy to follow macrophages having phagocytosed A549 cells.

B. Quantification of the capacity of macrophages expressing the indicated CAR construct to phagocytose A549 cells expressing or not CD19. The percent of phagocytosis was defined as the % of GFP+ events within the CD45+ population and plotted for each macrophage population. Each dot represents one donor.

Note that all CAR Macrophages, regardless of the CAR intracellular domain, phagocytosed CD19+ cells but not CD19− cells.

FIG. 3. CAR Macrophages are not able to control the growth of tumor spheroids in co-culture assays

IncuCyte based spheroid growth assay of A549-GFP-CD19 co-cultured with CAR Macrophages at a 16:1 E:T ratio. CAR Macrophages were prepared as for FIG. 1. 1000 tumor cells were seeded with 16000 Macrophages expressing the indicated CAR construct in Ultra-Low Attachment 96-Well Plates, resulting in the formation of tumor spheroids which growth in 3D. GFP intensity was followed by time-lapse microscopy every 3h for 96h. Mean of 3 donors+/−SD are displayed.

FIG. 4. Tumor growth control capacity of CAR Macrophages (CAR-MØ) in 3D

IncuCyte-based spheroid growth assay of MDA-MB-231GFP⁺CD19⁺ (A) or A549GFP⁺CD19⁺ (B) cells co-cultured with various CAR-MØ. CAR-MØ were prepared as for FIG. 1. At day-3, 10³ A549 or 10³ cells were seeded in Ultra-Low Attachment 96-well plates resulting in the formation of tumor spheroids growing in 3D. 3 days later 8·10³ untransduced macrophages or CAR-MØ were added or established spheroids. GFP intensity was followed by time-lapse microscopy every 3h for 96h. Mean of 3 donors+/−SD are displayed.

FIG. 5. Antigen stimulation of CAR Macrophages induce secretion of proinflammatory cytokines

Quantification of the indicated cytokine secreted by macrophages expressing CAR constructs upon co-culture with media alone, A549-GFP or A549-GFP-CD19 cells. CAR Macrophages were prepared as for FIG. 1 and were cultured at a 2:1 E:T ratio for 24h. Each dot represents one donor.

CAR Macrophages harboring a CD40 domain according to the invention secrete high levels of pro-inflammatory cytokines IL-6 and IL-8 upon co-culture with CD19+ cells and undergo a baseline secretion of TNFα in absence of antigen stimulation.

FIG. 6. CAR-monocytes can induce in vivo tumor regression

(A) NSG mice were injected intraperitoneally (IP) with 1·10⁶ MDA-MB-231 BFP-luc-CD19 cells. Mice were left untreated or injected IP with 7·10⁶ CAR Monocytes.

(B) Tumor size measured by bioluminescence using an IVIS system over 25 days.

### EXAMPLE 1: ENGINEERING MACROPHAGES FOR ANTI-TUMOR IMMUNITY

### Materials and Methods

### Cell Lines

A549 human lung tumor adenocarcinoma cell line and MDA-MB-231 human breast adenocarcinoma cell line were maintained in RPMI complete medium (Gibco™ Roswell Park Memorial Institute 1640 complemented with 10% fetal calf serum and 1% Gibco™ Penicillin-Streptomycin (Thermofischer)). HEK 293 FT cells were maintained in DMEM complete medium (Gibco™ Dulbecco's Modified Eagle Medium complemented with 10% fetal calf serum and 1% Penicillin-Streptomycin).

A549-GFP and A549-GFP-CD19 were obtained by lentiviral transduction with pWPXLd-GFP coding for GFP and with pCDH1-CD19 coding for hCD19. Transduced cell lines were FACS sorted to obtain homogenous cell populations.

### Primary Cells

Peripheral blood mononuclear cells (PBMC) were separated from plasmapheresis residues using Ficoll-Paque (GE Healthcare). Informed consent was obtained from all donors, and samples were deidentified prior to use in the study. Monocytes were isolated by CD14⁺ positive selection using CD14 magnetic microbeads (Miltenyi 130-050-201).

### Plasmid Construction and Virus Production

CAR constructs were cloned into pCDH1 lentiviral vector containing a puromycin resistance gene under the control of an EF1α promoter. All CAR constructs were expressed under the control of a CMV promoter.

Lentivirus were produced in HEK293 FT cells. Lentiviral vectors were co transfected with psPAX2 (2nd generation lentiviral packaging plasmid) and pMD2.G (encoding VSV-G) using PEI MAX® (Polysciences). Vpx-VLPs were produced in HEK293FT cells by transfection of pSIV3 and pMD2.G (S. Bobadilla et al., 2013)

After 18 h the media was replaced with fresh media to remove transfection reagent. Supernatants containing lentivector were collected 24 h after medium change and filtered with a 0.45 μm filter.

### Monocyte Transduction and Differentiation

CD14⁺ cells were transduced with lentivectors in presence of Vpx-VLPs and 4 μg/ml protamine. Monocytes were then allowed to differentiate in macrophages for 10 days in macrophage medium (RPMI+5% fetal calf serum+5% human serum+1% Penicillin-Streptomycin) with 50 ng/ml M-CSF in Corning® 100 mm Not TC-treated Culture Dish.

### Detection of CAR Expression

Human primary CAR Macrophages were stained with a rhCD19-Atto647 conjugated protein (R&D systems, ATM9269). Cells were harvested with StemPro Accutase Cell Dissociation Reagent (ThermoFischer) and washed with PBS then stained with rhCD19 Atto647 conjugated protein for 30 min at 4° C. Human FcBlock™ (BD Biosciences) was added during the staining. Cells were analyzed with FACS using a BD Verse.

### FACS-Based Phagocytosis Assay

1×10⁵ CAR Macrophages were co-cultured with 1×10⁵ A549-GFP or A549-GFP-CD19 cells for 3 h at 37° C. Cells were harvested with Accutase and stained with anti-CD45-Alexa700 (Biolegend) antibody in presence of FcBlock™ (BD Biosciences) and analyzed with FACS using a Bio-Rad ZE5. The percent of GFP+ events within the CD45+ population was plotted as the percentage of phagocytosis.

### IncuCyte-Based Spheroid Growth Assay

1×10³ tumor cells were seeded with 1.6×10⁴ macrophages in Corning® Costar® Ultra-Low Attachment 96-Well Plates (Merck). GFP fluorescence was then followed and measured on several days with Incucyte® S3 Live-Cell Analysis System (Essenbioscience). GFP intensity analysis was performed with IncuCyte software. After background removing, green objects were defined with a threshold. Total GFP⁺ integrated intensity is the sum of pixels belonging to all green objects.

### Cytokine Secretion Assay

1.5×10⁵ CAR Macrophages were co-cultured either with media, or 7.5×10⁴ A549-GFP or 7.5×10⁴ A549-GFP-CD19 for 24 h at 37° C. in Corning® Costar® Not Treated 12-well. Supernatant was collected and clarified by centrifugation. IL-6, IL-8 and TNFα concentrations were measured by cytometric bead array (Human Flex Set BD™ CBA, 558276, 558277, 558273) according to manufacturer's instructions.

### Results

### 1. Generation of CAR Expressing Macrophages

The inventors developed CAR constructs for macrophages to induce their activation only upon their encountering of a tumor-specific antigen, here CD19. Thus, all the CAR constructs contain an anti-CD19 single chain antibody (scFv) fused with the hinge and the transmembrane domains of CD8. The inventors included here the intracellular domain of CD40. Two different CAR constructs were built, see FIG. 1A. CAR Stop has no intracellular domain (it is a negative control for signal transduction), CAR CD40 is the CAR according to the invention.

The 2 CAR constructs were cloned into a lentiviral vector and used to transduce, together with pseudo particles carrying the Vpx SIV accessory protein, human primary monocytes.

Cells were then allowed to differentiate for 10 days in culture with M-CSF without antibiotic selection. The resulting 2 types of transduced macrophages, named CAR-Mϕ, displayed high surface expression of their CAR as assayed by FACS using recombinant CD19 ectodomain labeled with a fluorophore. Importantly, i) for each donor tested, at least 90% of CAR-Mϕ expressed the CAR construct at their surface (FIG. 1B), and ii) these high rates of transduction were obtained without any antibiotic selection.

### 2. Phagocytosis Capacity of CAR-MØ in 2D

To evaluate the capacity of the CAR-MØ to phagocytose tumor cells, the inventors had to generate appropriate target cells. Starting from the A549 lung adenocarcinoma cell line, they established cells stably expressing CD19 and GFP by lentiviral transduction. Incubation of the 2 different CAR-expressing MØ with A549GFP⁺CD19⁺ cells for 2 h at 37° C., but not with A549GFP⁺CD19−, led to the formation of double positive single cells that were both GFP⁺ and CD45⁺ as seen by flow cytometry (FIG. 2). In contrast, no double positive cells were detected when they used non-transduced MØ instead of CAR-MØ. These results suggest that in all likelihood, primary human anti-CD19 CAR-MØ can perform antigen specific phagocytosis of A549GFP⁺CD19⁺ cells, regardless of the presence of the CAR intracellular domain they possess (FIG. 2).

### 3. CAR-MØ are not Able to Control the Growth of Tumor Spheroids in Co-Culture Assays

Next, the inventors tested the capacity of CAR-MØ to impact the growth in 3D of spheroids of A549 cells over a 4-day period, by co-culture of A549 cells and CAR-MØ. The results do not show any significant difference between CAR Stop and CAR CD40 on the control of the growth of A549GFP⁺CD19⁺ tumor spheroids (FIG. 3). Importantly, CAR-MØ lacked anti-tumor activity against CD19− tumor spheroids.

### 4. Tumor Growth Control Capacity of CAR-MØ in 3D

The inventors tested the antitumor capacity of CAR-macrophages under more challenging conditions. They added CAR MØ to established tumor cell spheroids. They formed spheroids from 1000 A549-GFP-CD19 cells or 1000 MDA-MB-231-GFP-CD19 cells and added 3 days later 8000 untransduced macrophages or 8000 CAR MØ (FIG. 4). Surprisingly, addition of the CD40 domain as intracellular domain of the CAR increased the ability to control tumor growth in both tumor models.

### 5. Cytokine Production by CAR-MØ

The inventors next tested the polarization of CAR-MØ upon co-culture with target cells expressing or not CD19. CAR-MØ harboring a CD40 domain secreted high levels of the pro-inflammatory cytokines IL-6 and IL-8, upon co-culture with A549CD19⁺ cells and undergo a baseline secretion of TNFα in absence of antigen stimulation. The CAR-Stop MØ and the untransduced MØ hardly produced any cytokines in all conditions (FIG. 5). Thus, exposure of CD40 domain-containing CAR-MØ to CD19⁺ cells induced a shift towards a pro-inflammatory phenotype.

Thus, this work is the first demonstration of the efficiency of a CAR in macrophages containing a cytoplasmic domain of CD40, the impact on cytokine expression and on tumor regression.

### 6. Anti-Tumor Activity of CAR-MØ In Vivo

To evaluate the anti-tumor activity of CAR-MØ in vivo, the inventors used the human breast carcinoma MDA-MB-231 cell line to generate cells expressing CD19, BFP and luciferase by lentiviral transduction. Thus, MDA-MB-231 BFP-luc+CD19+ cells were injected intraperitoneally (i.p.) into immunodeficient NSG mice. The same day mice received an i.p. injection of 7·10⁶ CAR CD40-monocytes (invention) or CAR-CD3 monocytes (comparative). The growth of the tumors were regularly monitored over a 25-day period.

The inventors observed that the CAR-CD3 monocytes were unable to control tumor growth (FIG. 6). In contrast, 4 out of 6 mice of the group which received CAR-CD40 monocytes of the invention demonstrated a marked reduction in their tumor burden.

The inventors concluded that transduction of monocytes with the CAR-CD40 encoding vector according to the invention results in cells able to induce tumor regression in immunodeficient mice (FIG. 6).

1. A modified cell comprising a chimeric antigen receptor (CAR), wherein said CAR comprises:
an extracellular antigen-binding domain which binds to a tumor antigen or an antigen present on cells of the tumor microenvironment (TME);
optionally a hinge domain;
a transmembrane domain; and
an intracellular signaling domain comprising the CD40 cytoplasmic tail;
and wherein said modified cell is a myeloid cell.

2. The modified cell according to claim 1, wherein the cell is a monocyte, a macrophage or a dendritic cell.

3. A modified induced pluripotent stem cell (iPS) or hematopoietic stem cell (HSC) comprising a CAR, wherein said CAR comprises:
an extracellular antigen-binding domain which binds to a tumor antigen or a TME antigen;
optionally a hinge domain;
a transmembrane domain; and
an intracellular signaling domain comprising the CD40 cytotail.

4. The modified cell according to claim 1, wherein the extracellular antigen-binding domain is chosen from CD19, MUC16, MUC1, CA1X, carcinoembryonic antigen (CEA), CD8, CD7, CD 10, CD20, CD22, CD30, CLL1, CD33, CD34, CD38, CD41, CD44, CD49f, CD56, CD74, CD133, CD138, EGP-2, EGP-40, EpCAM, erb-B2,3,4, FBP, Fetal acetylcholine receptor, folate receptor-a, GD2, GD2Ac, GD3, ITER-2, hTERT, IL-13R-a2, K-light chain, KDR, LeY, LI cell adhesion molecule, MAGE-A1, Mesothelin, ERBB2, MAGEA3, p53, MARTI, GPI00, Proteinase 3 (PR1), Tyrosinase, Survivin, EphA2, NKG2D ligands, NY-ES0-1, oncofetal antigen (h5T4), PSCA, PSMA, ROR1, TAG-72, VEGF-R2, WT-I, BCMA, CD123, CD44V6, NKCS1, EGF1R, EGFR-VIII, CD99, CD70, ADGRE2, CCR1, LILRB2, PRAME, CCR4, CD5, CD3, TRBC1, TRBC2, TIM-3, Integrin B7, ICAM-I, CD70, Tim3, CLEC12A, ER, human telomerase reverse transcriptase (hTERT), mouse double minute 2 homolog (MDM2), cytochrome P450 1B1 (CYP1B), HER2/neu, p95HER2, Wilms' tumor gene 1 (WT1), livin, alphafetoprotein (AFP), prostate-specific membrane antigen (PSMA), cyclin (DI), mesothelin, B-cell maturation antigen (BCMA) and tumor-associated calcium signal transducer 2 (TROP2), preferably the tumor antigen is CD19.

5. The modified cell according to claim 1, wherein the extracellular antigen-binding domain is an anti-CD19 binding domain, preferably an anti-CD19 scFV.

6. The modified cell according to claim 1, wherein the intracellular signaling domain consists of the CD40 cytoplasmic tail.

7. The modified cell according to claim 1, wherein the CAR comprises, from its N-terminal end to its C-terminal end:
an extracellular antigen-binding domain of sequence SEQ ID NO:1,
optionally a hinge domain of sequence SEQ ID NO:2,
a transmembrane domain of sequence SEQ ID NO:3, and
an intracellular signaling domain of sequence SEQ ID NO:4.

8. The modified cell according to claim 1, wherein the cell comprises an additional vector, said vector comprising a sequence coding for a gene of interest under the control of a cytokine specific promoter.

9. A pharmaceutical composition comprising the modified cell of claim 1 and a pharmaceutical acceptable carrier.

10. A method of treatment of cancer or an inflammatory disease comprising administering to a subject in need thereof the modified cell according to claim 1.

11. The method of claim 10, wherein the cancer is a solid tumor.

12. A method of treatment of cancer or an inflammatory disease comprising the simultaneous, separate or sequential administration to a subject in need thereof of a combined preparation of products containing a modified cell according to claim 1 and a CAR-T cell.

13. A method of treatment of cancer or an inflammatory disease comprising the simultaneous, separate or sequential administration to a subject in need thereof of a combined preparation of products containing a modified cell of claim 1 and an Immune Checkpoint Inhibitor.

14. A method for manufacturing a modified cell comprising a CAR according to claim 1, the method comprising:
providing at least one cell chosen from isolated myeloid cells, iPS and isolated HSC; and
transducing said cell with a vector comprising a nucleic sequence coding for said CAR, preferably a lentiviral vector.

15. The method of claim 14, which further comprises introducing into said cell an additional vector, said vector comprising a sequence coding for a gene of interest under the control of a cytokine specific promoter.

16. In vitro method for assessing the targeted effector activity, preferably antigen-dependent phagocytosis of tumor cells, of a modified cell according to claim 1, comprising:
a) Culturing at least one tumor cell line or at least one tumor cell from a primary tumor in ultra-low binding plate, so that all cell lines or cells growth in a spheroid form;
b) Following the growth of the spheroid cultured cell lines or cells by time-lapse microscopy, in order to obtain at least one 3D spheroid;
c) Adding the modified cell to the 3D spheroid obtained in step b), to obtain a 3D spheroid culture; and
d) Analyzing a sample of the 3D spheroid culture obtained in step c) and/or the supernatant thereof, for example by time-lapse microscopy or supernatant chemical analysis.
